(19) United States
(12) Patent Application Publication
(10) Pub. No.: US 2015/0305892 A1
(43) Pub. Date: Oct. 29, 2015

(54) Methods and Devices for Treating a Bodily Lumen with In Situ Generated Structural Support

(71) Applicant: Abbott Cardiovascular Systems Inc., Santa Clara, CA (US)

(72) Inventors: Syed Hossainy, Hayward, CA (US); Krishna Sudhir, Santa Clara, CA (US); John Stankus, Campbell, CA (US); Mikael Trollsas, San Jose, CA (US)

(21) Appl. No.: 14/262,516

(22) Filed: Apr. 25, 2014

(51) Int. Cl. A61F 2/50; A61L 31/04; A61L 31/06; A61F 2/82; A61L 31/14

(52) U.S. Cl. CPC A61F 2/50; A61F 2/82; A61L 31/148; A61L 31/06; A61L 31/048; A61F 2210/0004; A61F 2210/0085

(57) ABSTRACT

A bodily lumen, such as a blood vessel, can be treated by forming a structural support in situ within the bodily lumen. This can be done by ejecting a formulation that includes a polymer that solidifies over a period of time, such as due to DMSO exchange or photocrosslinking. This can also be done by cooling a formulation until it freezes in situ. The structural support can also be made from a plaque which is already present in the bodily lumen. The plaque can be compressed by a balloon catheter and cooled so that it hardens and thereby forms the structural support. The bodily lumen can also be treated using a preformed structural support made of ice, for example frozen isotonic saline, or a fast degrading polymer, such as PEG. The preformed support is created outside of the bodily lumen, and then transported on a catheter to the treatment zone.

### FIELD

This application relates generally to medical devices and methods and, more particularly, to medical devices and methods for treating a bodily lumen using structural supports.

### INCORPORATION BY REFERENCE

All publications and patent applications mentioned in this specification are herein incorporated by reference to the same extent as if each individual publication or patent application was specifically and individually indicated to be incorporated by reference.

### BACKGROUND

Endoluminal prostheses or endoprostheses are medical devices adapted to be implanted in a human or veterinary patient. Stents are a type of endoprosthesis which are deployed in a blood vessel, urinary tract, bile duct, or other bodily lumen to provide structural support and optionally to deliver a drug or other therapeutic agent. Stents are generally cylindrical and function to hold open and sometimes expand a segment of the bodily lumen. Stents are often used in the treatment of atherosclerotic stenosis in blood vessels. Stents are often delivered to a desired location while in a reduced configuration having a smaller diameter than when fully deployed. The reduced configuration allows the stent to be navigated through very small passageways, such as coronary vessels and other bodily lumen. A crimping process is performed to place the stent in a reduced configuration. The stent can be crimped onto a catheter that can then be maneuvered over a guidewire that leads to a region of the anatomy at which it is desired to deploy the stent. The passageway through which the stent is maneuvered is often tortuous, so the stent should be capable of longitudinal flexibility. Once the stent has reached the desired deployment location, the stent is allowed to self-expand or is forcibly expanded by a balloon to an enlarged configuration. After deployment, the stent should maintain its enlarged configuration with minimal recoil back to its reduced configuration. All these functional requirements are taken into account in the structural design of a stent.

Due to the mechanical stresses involved, crimping and subsequent expansion during deployment pose significant challenges in the structural design of certain endoprostheses. It would be desirable to have an endoprostheses that can be implanted without having to be subjected to mechanical stresses during crimping and subsequent expansion.

In addition, endoprostheses are often manufactured in a limited number of predetermined sizes and shapes. However, none of the predetermined sizes and shapes may be optimal for a particular situation because of variations in the size of patients, variations in anatomy, variations in the shape of lesions, etc. Thus, it would be desirable to have an endoprostheses that can be customized in terms of size and configuration according to need.

### SUMMARY

Described herein are methods and devices for treating a bodily lumen.

In various aspects, a method comprises forming a structural support in situ within a treatment zone of a bodily lumen.

In additional aspects, forming the structural support includes ejecting a formulation through a lumen of a catheter and onto a wall of the treatment zone, followed by solidifying the ejected formulation on the wall of the treatment zone.

In additional or alternative aspects, the formulation includes a bioresorbable polymer and optionally a therapeutic agent.

In additional or alternative aspects, the formulation includes at least one photocrosslinkable polymer. Solidifying of the ejected formulation includes delivering optical radiation to the photocrosslinkable polymer in the treatment zone. The at least one photocrosslinkable polymer increases in hardness as a result of the optical radiation.

In additional or alternative aspects, the formulation includes isotonic saline, and solidifying the ejected formulation includes freezing the isotonic saline in the treatment zone. Freezing of the isotonic saline includes cooling the catheter to a temperature above a damage threshold of tissue in the treatment zone.

In additional or alternative aspects, forming of the structural support includes cooling plaque present in the treatment zone, and the cooling causes the plaque to increase in hardness.

In additional or alternative aspects, forming the structural support further includes compressing the plaque before or during cooling of the plaque.

In additional or alternative aspects, cooling of the plaque causes the plaque to increase in hardness without cyroablating tissue surrounding the plaque.

In additional or alternative aspects, forming the structural support includes introducing an additive to plaque present in the treatment zone, and the additive causes formation of a hardened composite of the plaque and the additive.

In additional or alternative aspects, forming the structural support includes pressing a plurality of bioabsorbable polymeric nanoparticles onto plaque present in the treatment zone, and the bioabsorbable polymeric nanoparticles cause the plaque to increase in hardness.

In additional or alternative aspects, forming the structural support includes anchoring a plurality of rivets into plaque present in the treatment zone, and the rivets cause the plaque to increase in hardness.

In various aspects, a method comprises cooling and structurally supporting a treatment zone of a bodily lumen, wherein the cooling and supporting are performed simultaneously.

In additional aspects, the cooling and structurally supporting include forming a structural support in situ within the treatment zone.

In additional or alternative aspects, the cooling and structurally supporting include depositing into the treatment zone a structural support that was formed outside of the treatment zone, and the structural support is made of a frozen formulation capable of melting completely in the bodily lumen within about 30 minutes.

In additional or alternative aspects, the method further comprises freezing the formulation in a mold to form the structural support, and then transporting structural support on a catheter to the treatment zone.

In additional or alternative aspects, the cooling of the treatment zone is performed without cryoablation of tissue in the treatment zone.

In various aspects, an endoprosthesis comprises a structural support made of a frozen formulation having a freezing temperature below about 0° C.

In additional aspects, the frozen formulation is frozen isotonic saline.

In various aspects, a system for treating a bodily lumen comprises any one of the endoprosthesis above, and a catheter configured to carry the structural support at a temperature at or below the freezing temperature.

In various aspects, an endoprosthesis comprises a structural support made of a bioresorbable formulation including a polymer selected from the group consisting of PEG and a PEG-based polymer.

In additional aspects, the structural support is made of a sheet of material that contains the bioresorbable formulation as a first layer between second and third layers, and the second and third layers biodegrade completely over a period of time that is greater than that of the bioresorbable formulation.

In additional aspects, the second layer is made of poly(lactic acid), and the third layer is made of polyglycolic acid.

In various aspects, a method comprises depositing any one of the structural supports above, and allowing the structural support to biodegrade completely at a time after the depositing, the time being within the range of about 7 days to about 30 days.

In various aspects, a catheter comprises an inflatable balloon, and a plurality of bioabsorbable rivets carried on an outer surface of the balloon. Each rivet includes a tip and a base wider than the tip. The tips face outward from the outer surface. Each rivet is configured to detach from the balloon when the tip is pressed into tissue.

In additional aspects, the catheter further comprises a bioabosrbable mesh covering the outer surface of the balloon. The rivets are disposed in the mesh, and the mesh is configured to detach from the balloon together with the rivets when the tips are pressed into tissue.

The features and advantages of the invention will be more readily understood from the following detailed description which should be read in conjunction with the accompanying drawings.

### BRIEF DESCRIPTION OF THE DRAWINGS

FIG. 1 is a flow diagram showing a method for treating a bodily lumen by ejecting a formulation into the bodily lumen.

FIG. 2 is a partial longitudinal cross-sectional view showing a catheter for forming a structural support in situ within a bodily lumen.

FIG. 3 is a partial longitudinal cross-sectional view showing a distal end segment of the catheter of FIG. 2 within a bodily lumen.

FIG. 4A is a longitudinal cross-sectional view showing a formulation being ejected from the distal end segment.

FIG. 4B is an axial cross-sectional view showing the formulation on the wall of the bodily lumen.

FIGS. 5 and 6 are isometric views showing structural supports created from hardening of an ejected formulation.

FIGS. 7 and 8 are a partial longitudinal cross-sectional views showing catheters for forming a structural support in situ within a bodily lumen.

FIG. 9 is a partial longitudinal cross-sectional view showing a balloon at a distal end segment of a catheter for forming a structural support in situ within a bodily lumen.

FIG. 10 is longitudinal view showing a balloon of a catheter for forming a structural support in situ within a bodily lumen.

FIG. 11 is an isometric view of a structural support formed in situ by the balloon of FIG. 10.

FIGS. 12 and 13 are partial longitudinal cross-sectional views showing balloons of catheters for forming structural supports in situ within bodily lumens.

FIG. 14 is a flow diagram showing a method for treating a bodily lumen by compressing and hardening plaque in a bodily lumen.

FIG. 15A-15C are partial longitudinal cross-sectional views showing a catheter that compresses and hardens plaque in a bodily lumen and showing the plaque after removal of the catheter.

FIGS. 15D-15F are detailed views showing a portion of a balloon outer surface of the catheter of FIG. 15A.

FIG. 16 is a partial longitudinal cross-sectional view showing a catheter for freezing a formulation in situ within a bodily lumen.

FIG. 17 is a flow diagram showing a method for treating a bodily lumen by depositing a cooled structural support in a bodily lumen.

FIG. 18 is a partial longitudinal cross-sectional view showing a catheter for transporting a cooled structural support into a bodily lumen.

FIGS. 19A and 19B are cross-sectional views showing molds for freezing a formulation to make cooled structural supports to be transported into bodily lumens.

FIG. 20 is a partial cross-sectional view showing a sheet of material for use in making a preformed structural support.

### DETAILED DESCRIPTION OF EXEMPLARY EMBODIMENTS

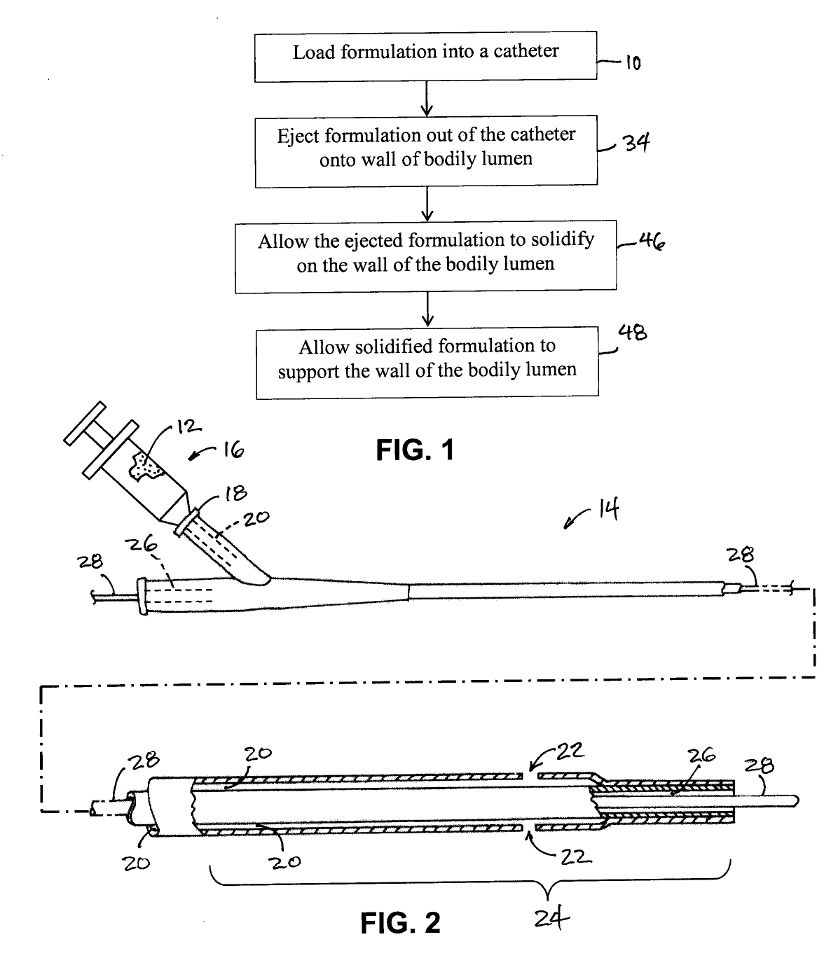

Referring now in more detail to the exemplary drawings for purposes of illustrating embodiments, wherein like reference numerals designate corresponding or like elements among the several views, there is shown in FIG. 1 exemplary method of forming an endoprosthesis in situ within a bodily lumen. In this context, “in situ” means that the endoprosthesis does not exist before its formation within the bodily lumen. The bodily lumen can for example, without limitation, be a blood vessel, urinary tract, or bile duct.

In the following description of the method of FIG. 1, reference will be made to other figures that illustrate an exemplary catheter for performing the method. It is to be understood that the method of FIG. 1 is not limited to the illustrated catheter.

In block 10, formulation 12 is loaded into catheter 14 (FIG. 2) configured for insertion into the bodily lumen. Formulation 12 can be loaded in catheter 14 before, during, and/or after catheter 14 is inserted into the bodily lumen. Formulation 12 can be loaded using pump device 16, such as plunger-type syringe or motor powered pump, connected to formulation entry aperture 18 of the catheter. As formulation 12 is loaded, it travels through formulation lumen 20, which extends from formulation entry aperture 18 to one or more formulation outlet apertures 22. Formulation outlet apertures 22 are located in distal end segment 24 of the catheter.

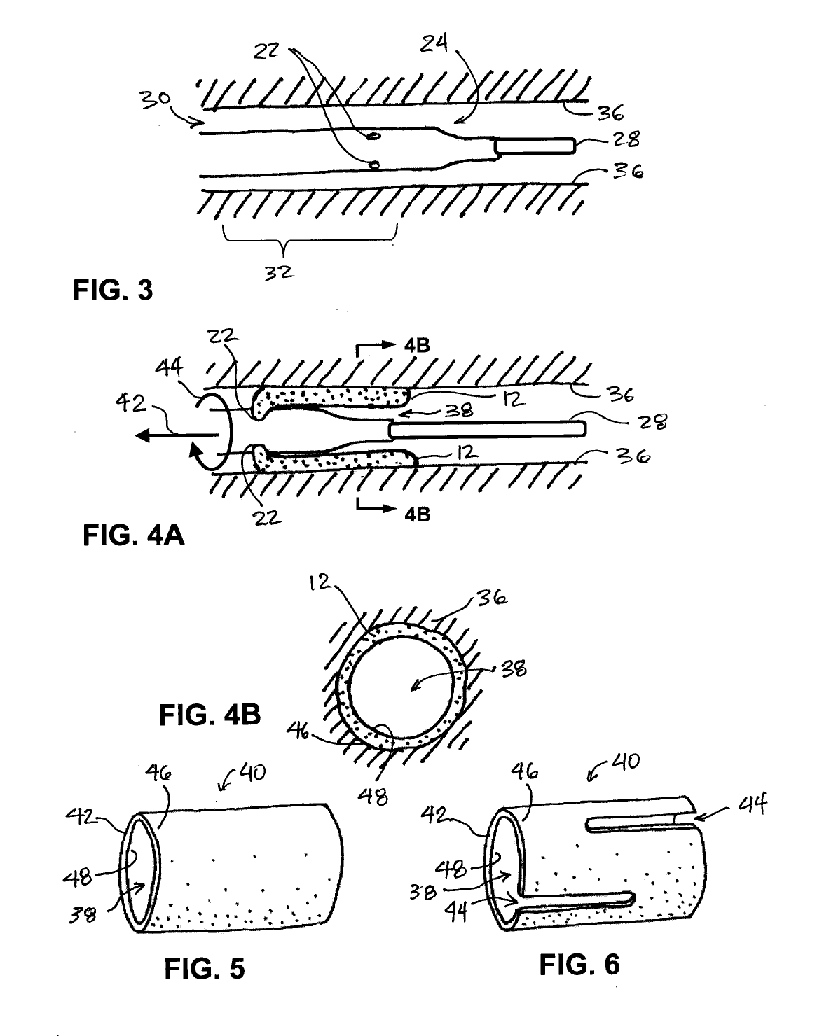

Catheter 14 includes guidewire lumen 26 which is configured to receive guidewire 28. Guidewire 28 can first be inserted into bodily lumen 30 and navigated to treatment zone 32 (FIG. 3) within the bodily lumen. For example and without limitation, treatment zone 32 can be any of a stenosis, plaque, and weaken or injured segment of a blood vessel or other bodily lumen. Alternatively, no guidewire is used and the catheter for delivering formulation 12 need not have a guidewire lumen.

In block 34 (FIG. 1), only when distal end segment 24 is within treatment zone 32, formulation 12 is ejected out of exemplary catheter 14 via formulation outlet apertures 22. As formulation 12 exits outlet apertures 22, formulation 12 is applied onto wall 36 of bodily lumen 30 (FIGS. 4A and 4B). Formulation 12 is in a soft state inside catheter 14 and when deposited on wall 36. Formulation 12 can be ejected onto wall 36 by continued pumping of formulation 12 into formulation entry port 18 (FIG. 2) by pump device 16. Alternatively, formulation 12 can be ejected onto wall 36 by pumping a purging liquid into formulation entry port 18 using a second pump device. The second pump device contains the purging liquid and is connected to formulation entry port 18. The purging liquid would push formulation 12 contained in formulation lumen 20 out of outlet apertures 22. The purging liquid could be an aqueous solution (solution containing water) or non-aqueous solution (solution not containing water) depending on the type for formulation being used. In some embodiments, the type of purging liquid used does not cause formulation 12 to solidify within catheter 14.

Formulation 12 is ejected in a controlled manner so that formulation 12 does not occlude bodily lumen 30. The amount of formulation that ejected is limited so that the formulation does not form a plug that completely obstructs bodily lumen 30. The amount that is ejected can be controlled by monitoring the volume of formulation 12 or purging liquid that is being pumped into formulation entry port 18. The amount that is ejected can be controlled by monitoring, through the use of one or more sensors, hydraulic pressure or flow rate at formulation entry port 18.

As formulation 12 exits outlet apertures 22, formulation 12 is applied onto wall 36 of bodily lumen 30 such that fluid passageway 38 is maintained between deposits of formulation 12. For example, catheter 14 can be pulled axially in the direction of arrow 42 while formulation 12 is ejected out of outlet apertures 22 in order to apply formulation 12 across the entire axial length of treatment zone 32, as shown in FIG. 4A. In addition or alternatively, catheter 14 can be rotated about its axis, such as in the direction of arrow 44, while formulation 12 is ejected out of outlet apertures 22 in order to apply formulation 12 across the entire circumference of wall 36, as shown in FIG. 4B. In FIG. 4B, catheter 14 and guidewire 28 have been removed from the bodily lumen.

In block 46 (FIG. 1), deposits of formulation 12 on wall 36 are allowed to solidify. Catheter 14 is pulled out of bodily lumen 30 before, during, or after solidification of formulation 12. In block 48, the solidified formulation remains in place within treatment zone 32 and supports wall 36 after catheter 14 is pulled out. After solidification, fluid passageway 38 remains to allow blood or any other body fluid to pass through treatment zone 32.

In some embodiments, the solidified formulation can have the shape of tube 40 (FIG. 5) having cylindrical wall 42 without fenestrations. The term “fenestrations” refers to holes or gaps that pass completely through a wall. Alternatively, the solidified formulation can have the shape of tube 40 having cylindrical wall 42 with fenestrations 44 (FIG. 6). Fenestrations 44 can have shapes other than what is illustrated. Wall 42 has abluminal surface 46 and luminal surface 48. The term “luminal surface” refers to the radially inward facing surface or the surface that faces toward fluid passageway 38. The term “abluminal surface” refers to the radially outward facing surface or the surface that faces away from fluid passageway 38. Abluminal surface 46 contacts wall 36 of bodily lumen 30.

The solidified formulation can be temporary. For example, the solidified formulation can be bioresorbable. The terms “biodegradable,” “bioresorbable,” “bioabsorbable,” and “bioerodable” are used interchangeably and refer to materials, such as but not limited to, polymers, that are capable of being completely degraded, eroded, and/or dissolved when exposed to bodily fluids such as blood and can be gradually resorbed, absorbed, and/or eliminated by the body. The processes of breaking down and absorption of the polymer can be caused by, for example, hydrolysis and metabolic processes.

In some embodiments, formulation 12 includes one or more polymers and dimethyl sulfoxide (DMSO). The polymers can be bioresorbable. DMSO allows the mixture to flow through formulation lumen 20. After ejection of formulation 12 out of outlet apertures 22 and onto wall 36, DMSO (contained within the deposits of formulation on wall 36) exchanges with an aqueous medium present in treatment zone 32 of bodily lumen 30. The aqueous medium can be introduced into treatment zone 32 before, during, and/or after formulation 12 is ejected out of outlet apertures 22. As a result of the exchange, the polymers solidify on wall 36.

For example, formulation 12 includes a blend of polycaprolactone (PCL), DMSO, and nanoparticles of poly(lactic acid) (PLA). After this blend of substances is ejected out of outlet apertures 22 and onto wall 36, DMSO exchanges with an aqueous medium present in treatment zone 32 of bodily lumen 30. As a result of the exchange, the PLA nanoparticles in PCL solidify and remain on wall 36. Forms of PLA include poly-L-lactide (PLLA) and poly-D-lactide (PDLA).

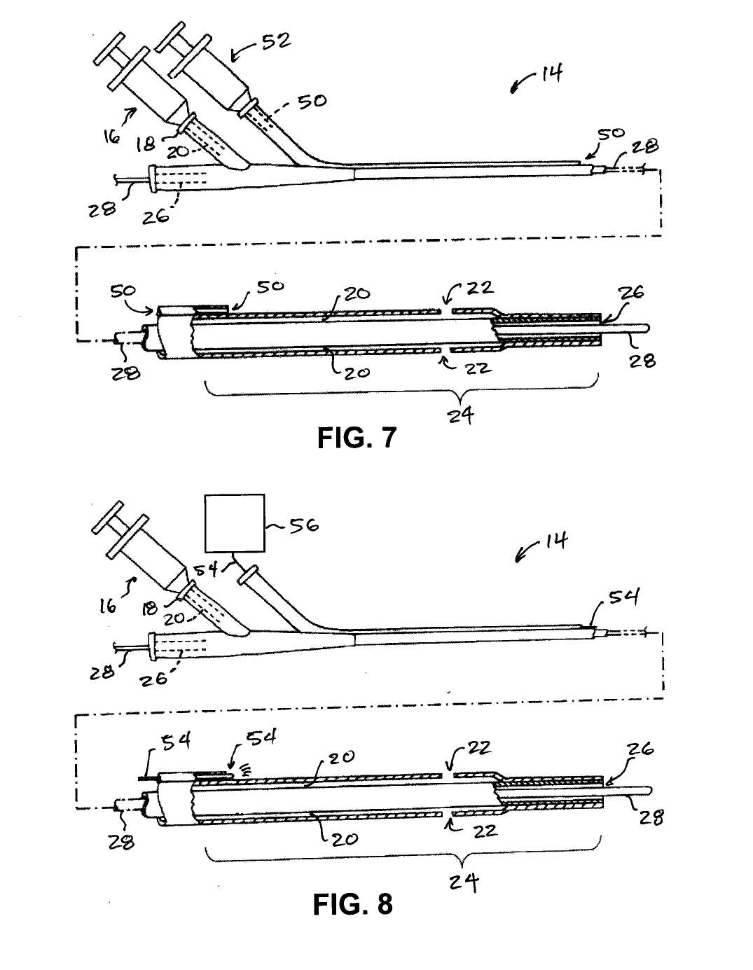

In some embodiments, as shown in FIG. 7, catheter 14 has supply lumen 50, in addition to formulation lumen 20 and guidewire lumen 26. Supply lumen 50 is configured to deliver an aqueous medium to treatment zone 32 to encourage hardening of formulation deposits on wall 36. The supply lumen may also be configured to suction or remove the aqueous medium from treatment zone 32. The aqueous medium can be provided and removed by pump device 52, such as plunger-type syringe or motor powered pump, coupled to supply lumen 50. Alternatively, another catheter (other than catheter 14 which delivers formulation 12) is used to provide the aqueous medium to treatment zone 32. The supply lumen can be implemented in any of the catheters described herein.

In some embodiments, formulation 12 includes a blend of one or more photocrosslinkable polymers and DMSO. The photocrosslinkable polymers are bioresorbable. DMSO facilitates flow of formulation 12 through formulation lumen 20. After ejection of formulation 12 out of outlet apertures 22 and onto wall 36, optical radiation is directed onto the deposits of formulation 12 on wall 36. The optical radiation has a wavelength that causes the polymers in formulation 12 to become crosslinked. The optical radiation can be delivered to the deposits of formulation 12 on wall 36 during and/or after formulation 12 is ejected out of outlet apertures 22.

For example, formulation 12 includes a blend of poly(lactic acid) diacrylate (PLADA), DMSO, and poly(ethylene glycol) diacrylate (PEGDA). During and/or after this blend of substances is ejected out of outlet apertures 22 and onto wall 36, optical radiation is directed onto the blend of substances on wall 36. The optical radiation has one or more wavelengths that cause the PLADA to crosslink and the PEGDA to crosslink. As a result of crosslinking, the blend hardens and remains on wall 36.

In some embodiments, as shown in FIG. 8, catheter 14 has fiber optic cable 54 configured to deliver optical radiation to treatment zone 32 to encourage hardening of formulation deposits on wall 36. The optical radiation can be provided by light source 56, such as laser light source or other type of light source, coupled to one end of fiber optic cable 54 which extends to distal end segment 24. The opposite end of fiber optic cable 54 emits optical radiation in the treatment zone. The fiber optic cable can be implemented in any of the catheters described herein. Alternatively, another catheter (other than catheter 14 which delivers formulation 12) is used to deliver the optical radiation to treatment zone 32.

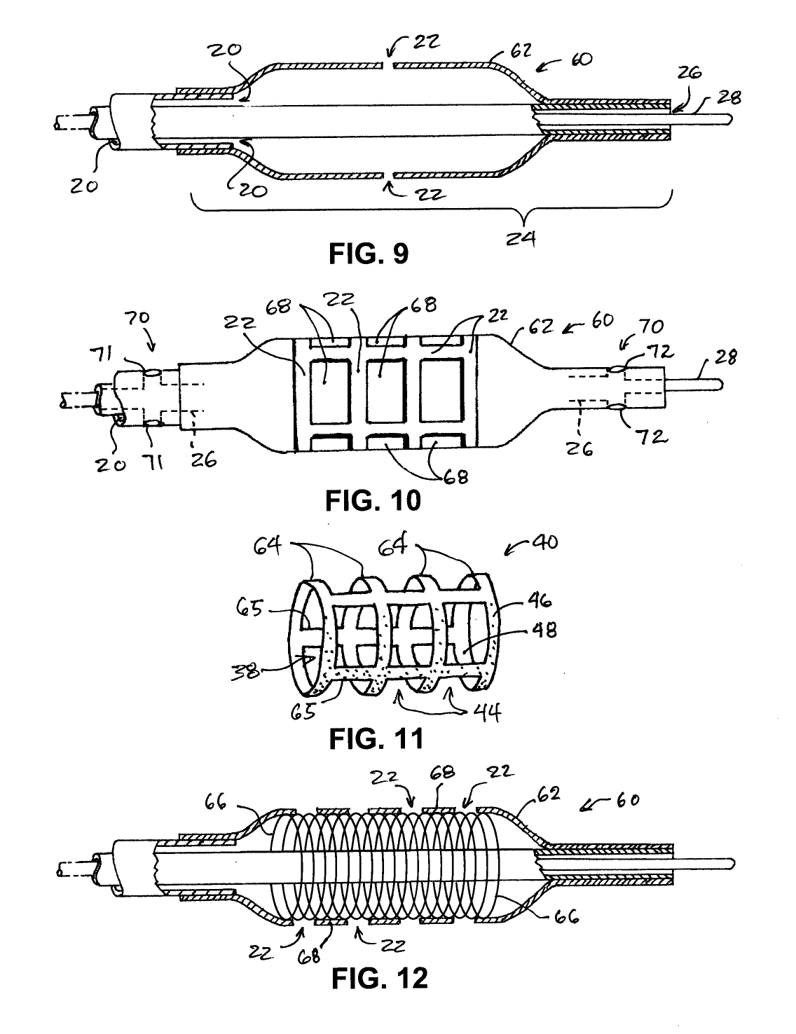

Further examples of ejecting formulation 12 onto wall 36 are described below. As shown in FIGS. 9, 10, 12 and 13, distal end segment 24 of catheter 14 can include inflatable balloon 60 configured to deliver formulation 12 to treatment zone 32. Ejection of formulation 12 (block 34 of FIG. 1) can include ejection from balloon 60. Balloon 60 can help ensure that formulation 12 is applied onto the wall of the bodily lumen and does not form a plug that completely obstructs the bodily lumen. Balloon 60 can also dilate treatment zone 32 of the bodily lumen.

In FIG. 9 for example, formulation lumen 20 can lead to the interior of balloon 60. Outlet apertures 22 can be formed through wall 62 of balloon 60. As formulation 12 is loaded into catheter 14 (block 10 of FIG. 1), formulation 12 begins to inflate balloon 60. After balloon 60 has inflated, formulation 12 is ejected out of outlet apertures 22 and onto the wall of the bodily lumen (block 34 of FIG. 1).

In FIG. 10 for example, outlet apertures 22 can be in the form of patterned slits formed through wall. Slits 22 may connect to form a repeating geometry, such as a plurality connected rings. Formulation 12 is ejected out of slits 22. Although the formulation may spread out slightly, deposits of formulation 12 on the wall of the bodily lumen can have the same pattern as slits 22. For example, some of the slits may intersect with each other to form a rectangle, triangle, or other geometric shape, and the formulation 12 (before and after it is solidified) on the wall of the bodily lumen can have a corresponding rectangle, triangle, or other geometric shape.

In FIG. 11 for example, the deposits of formulation 12 can form tube 40 with fenestrations 44. Fenestrations 44 can have shapes other than what is illustrated. Tube 40 includes connected rings 64 having widths that are the same as or greater than widths of patterned slits 22 of FIG. 10. Each ring 64 is connected by link 65 to an adjacent ring. Rings 64 and links 65 are made of solidified deposits of formulation 12. Rings 64 and links 65 function like a scaffold that can provide structural support to bodily lumen 30.

As shown in FIG. 12, balloon can include support 66, which can be filaments disposed on an interior surface of balloon wall 62. As mentioned above, slits 22 can connect with each other. Connected slits may form islands 68 (FIGS. 10 and 12) of balloon wall material. Islands 68 can be held in place by support 66. Support 66 does not block slits 22. When formulation 12 is being ejected out of slits 22, formulation 12 passes through gaps in support 66, such as gaps between filaments. Alternatively, support 66 can be disposed on an exterior surface of balloon wall 62.

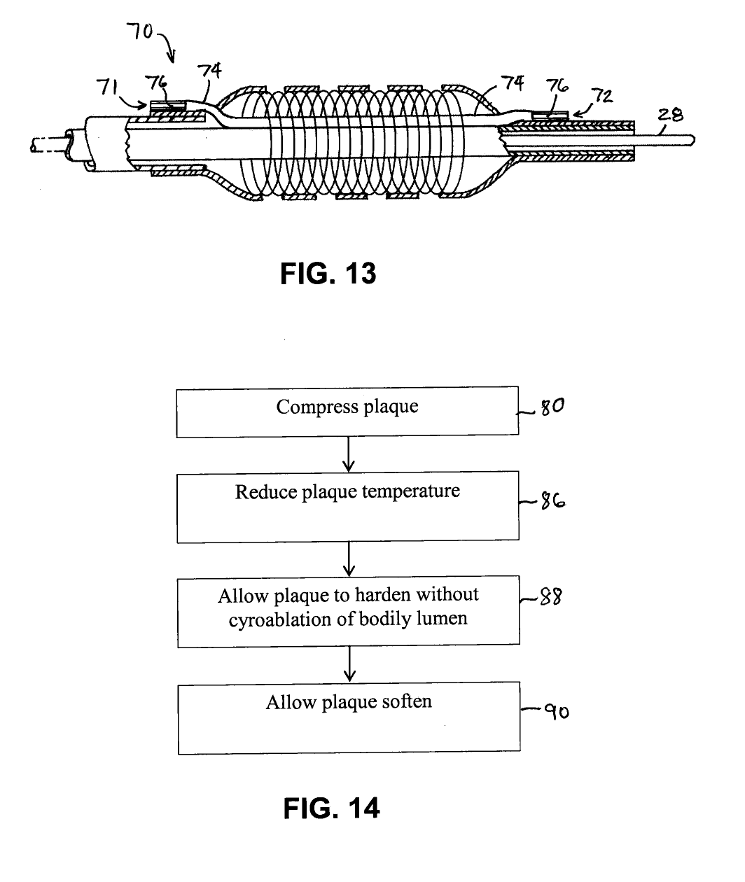

As shown in FIGS. 10 and 13, catheter 14 can have perfusion feature 70 configured to allow blood or other body fluid to pass across balloon 60 while balloon 60 is inflated. Perfusion feature 70 also allows passage of bodily fluids while formulation 12 is solidifying. In FIG. 10 for example, perfusion feature 70 includes apertures 71 and apertures 72 on the outer surface of catheter 14. Apertures 71 are located on one side of balloon 60. Apertures 72 are located on the other side of balloon 60. Apertures 71 and 72 lead to guidewire lumen 26. Blood or other bodily fluid can pass through balloon 60 via apertures 71 and 72 and guidewire lumen 26.

In FIG. 13 for example, perfusion feature 70 includes apertures 71 and apertures 72 at opposite ends of perfusion tube 74. Apertures 71 are located on one side of balloon 60. Apertures 72 are located on the other side of balloon 60. Apertures 71 and 72 lead to perfusion passageway 76 within perfusion tube 74. Blood or other bodily fluid can pass through balloon 60 via apertures 71 and 72 and perfusion passageway 76.

As described above, a formulation can be introduced into a bodily lumen, followed by solidification of the formulation, such that a tube or scaffold is formed in situ at the treatment zone of the bodily lumen. The tube or scaffold can provide temporary structural support until it is bioabsorbed.

As shown in FIG. 14, a method can be performed in which no formulation and no preformed prosthesis is introduced into a bodily lumen to provide structural support at the treatment zone. In the following description of the method of FIG. 14, reference will be made to other figures that illustrate an exemplary catheter for performing the method. It is to be understood that the method of FIG. 14 is not limited to the illustrated catheter.

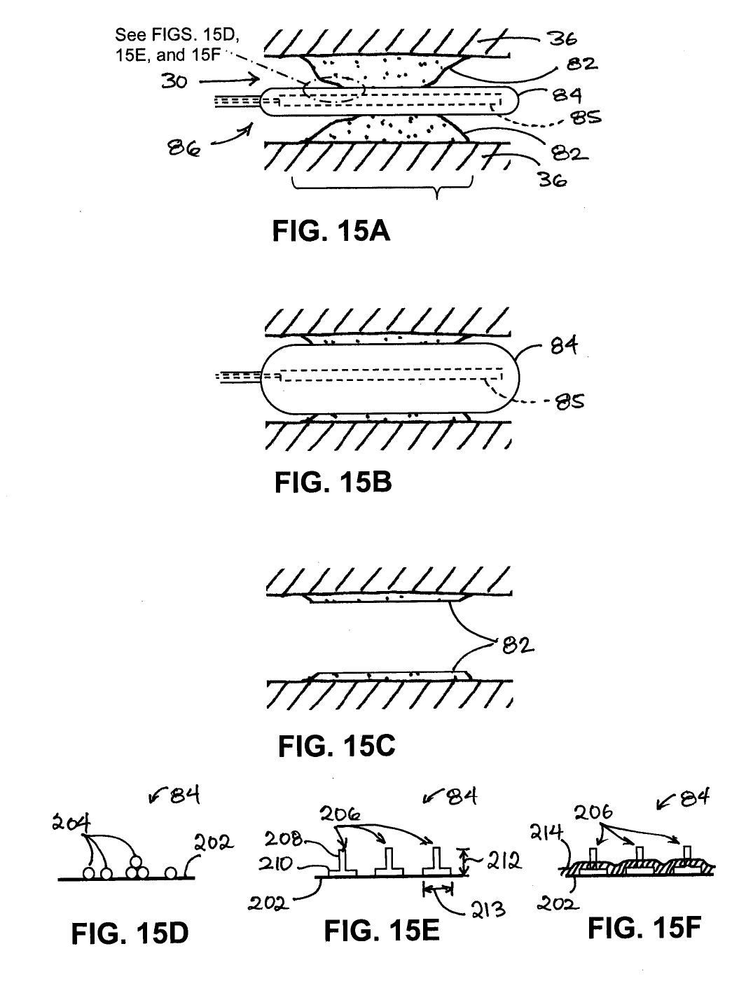

In block 80, plaque 82 on wall 36 is compressed against wall 36 by inflating balloon 84 at the distal end segment of catheter 86 (FIGS. 15A-15C). Compression allows for a decrease in the degree of blockage of the bodily lumen. Balloon 84 includes cooling and temperature control system 85. For example, refrigerant gas can be delivered by system 85 to the interior of balloon 84. The temperature of the refrigerant gas decreases as it expands within balloon 84, which makes contact with and compresses plaque 82 (FIG. 15B). As a result, in block 86, the temperature of plaque 82 is reduced. In block 88, continued reduction in temperature causes plaque 82 to harden or freeze.

Cryoablation systems are known in the art and need not be described herein. See, for example, Pub. Nos. 2001/0037081, 2009/0234345, and 2013/0345688. Details for cooling and temperature control from known cryoablation systems can be altered (such as by using a different refrigerant fluid) to make cooling and temperature control system 85 which is configured for cooling without cryoablation. In conventional cryoablation systems, however, the temperatures that are used are for the ablation of tissue, which means that the tissue is permanently destroyed or damaged. For example, temperatures below −70° C. are sometimes used in cryoablation.

Catheter 86 and its balloon are not configured for cryoablation. In the present method, balloon 84 is not allowed to drop to a temperature which will permanently destroy or damage walls of the bodily lumen. The temperature which will permanently destroy or damage tissue is referred to herein as the “damage threshold.” The damage threshold can vary depending upon the type of bodily lumen. Balloon 84 is allowed to drop to a temperature that is above the damage threshold and which will freeze or harden plaque 82. For example, balloon 84 is allowed to drop to a temperature between about 5° C. and about −30° C., or between about 5° C. and about −20° C., or between about 5° C. and about −15° C., or between about 1° C. and −5° C., or about −2° C.

When used as a modifier preceding a numerical value, the term “about” means plus or minus 10% of the numerical value. For example, “about −30° C.” encompasses −33° C. to −27° C., and “−20° C.” encompasses −22° C. to −18° C.

After plaque 82 hardens (block 88 in FIG. 14), the temperature of the plaque 82 is allowed to rise. For example, balloon 84 can be deflated and withdrawn from treatment zone 32 (FIG. 15C). In block 90, with continued warming, plaque 82 is allowed to soften and lose its hardness.

When hardened, the compressed plaque can provide temporary structural support to wall 36 of the bodily lumen. For example, plaque 82 may extend around the entire circumference of wall 36 to form a hardened tube, similar in shape to tube 40 in FIG. 5 or an irregularly shaped tube with or without fenestrations. The structural support provided by the compressed and hardened plaque may last from about 5 to about 30 minutes. The temporary structural support, in conjunction with reduction in biochemical pathways within the lesion during compression of plaque 82 at a low temperature, can create sustained patency of the flow area. The term “patency” is a condition in which the bodily lumen is not blocked or obstructed.

In the method of FIG. 14, a portion of plaque 82 can be removed during or before the plaque is allowed to harden (i.e., during or before any of blocks 80, 86 and 88).

The method of FIG. 14 can have other features described below. Any one or more of the features below can be implemented in combination with the descriptions of cooling above (performed together with blocks 80, 86, 88, and 90) or implemented without any cooling (performed with block 80 but without blocks 86, 88, and 90).

In the method of FIG. 14, balloon 84 (FIGS. 15A and 15B) can be configured to deliver an additive to plaque 82 which results in a hardened composite of plaque 82 and the additive. Balloon 84 (FIGS. 15A and 15B) can have the structure of balloons 60 (FIGS. 9, 10, 12, and 13). Before or during compression of plaque 82 (i.e., before or during block 80), the additive can be forced through a formulation lumen within balloon 84 and then applied onto plaque 82. Examples of additives include without limitation any one or a combination of: fibrin glue, isopropyl cyanoacrylate, carboxymethyl cellulose, hydroxypropyl methylcellulose, and small polymer fibers. The small polymer fibers can be made from bioresorbable polymers, such as PLLA, poly(lactic-co-glycolic acid) (PLGA), and PLA-co-PCL (a copolymer of PLA and polycaprolactone).

In the method of FIG. 14, balloon 84 (FIGS. 15A and 15B) can have outer surface 202 and bioabsorbable polymeric nanoparticles 204 (FIG. 15E) carried on outer surface 202. Bioabsorbable polymeric nanoparticles 204 can reduce the volume of plaque 82 and/or harden plaque 82. Examples of such bioaborbable polymeric nanoparticles include nanoparticles made of PLLA, PLGA, and PLLA-co-PDS (a copolymer of poly-L-lactide and polydioxanone). The size of nanoparticles 204 can be in the range of 75 nanometers (nm) to 1000 nm. When balloon 84 is inflated (block 80 of FIG. 14), nanoparticles 204 are transferred to plaque 82. Balloon outer surface 202 can be roughened and/or fiberous so that it can carry nanoparticles 204 and then transfer nanoparticles 204 to plaque 82 when balloon 84 is expanded and pressed into contact with plaque 82. After balloon 84 is deflated and removed from treatment zone 32, nanoparticles 204 remain embedded in plaque 82 and causes plaque 82 to reduce in volume and/or increase in hardness.

Optionally, balloon 84 can include a tubular sheath which covers balloon outer surface 202 when balloon 84 is being navigated through the bodily lumen. The sheath protects nanoparticles 204 and prevents nanoparticles 204 from detaching prematurely from balloon 84. When balloon 84 is near treatment zone 32, the sheath can be retracted or balloon 84 can be advanced out from the sheath and then inflated.

In the method of FIG. 14, balloon 84 (FIGS. 15A and 15B) can have outer surface 202 which carries rivets 206 (FIGS. 15E and 15F) made of PLLA or other bioabsorbable polymer material. Each rivet 206 has tip 208 and base 210 which is wider than tip 208. Rivets 206 have length 212 (measured from base 210 to tip 208) in the range of about 0.2 mm to about 1 mm. Base 210 can have width 213 of at least 0.2 mm, or at least 0.5 mm, or at least 1 mm. Tips 208 face outward from outer surface 202. Each rivet 206 is configured to detach from balloon 84 when tip 206 is pressed into tissue. When balloon 84 is expanded (block 80 of FIG. 14), tips 208 are pressed into plaque 82. Compressive force from balloon 84 causes rivets 206 to become anchored in plaque 82. After balloon 84 is deflated and removed from treatment zone 32, rivets 206 remain anchored in plaque 82. Rivets 206 cause plaque 82 to reduce in volume and/or increase in hardness.

As previously mentioned, balloon 84 can include a tubular sheath which covers balloon outer surface 202 when balloon 84 is being navigated through the bodily lumen. The sheath can protect rivets 206 and prevents rivets 206 from detaching prematurely from balloon 84.

Optionally, balloon outer surface 202 carries thin mesh 214 of fibers. Mesh 214 (FIG. 15F) covers balloon 84 and carries rivets 206. Mesh 214 can be fabricated as a thin flat sheet, and then rivets 206 can be embedded in mesh 214. Next, mesh 214 (together with rivets 206) can be wrapped around balloon 84. The fibers in mesh 214 are made of a bioabsorbable polymer that allows mesh 214 to completely biodegrade over a shorter period of time than rivets 206. Exemplary materials for mesh 214 include without limitation PEG-co-PBT (a copolymer of polyethylene glycol and polybutylene terephthalate), polyethylene glycol (PEG), polyvinylpyrrolidone (PVP), and PLGA. For PEG, the molecular weight is preferably high (e.g., greater than 100,000 g/mol). For PLGA, the glycolide content is preferably high. When balloon 84 is expanded (block 80 of FIG. 14), mesh 214 and rivets 206 are transferred to plaque 82. Compressive force from balloon 84 causes rivets 206 to become anchored in plaque 82. After balloon 84 is deflated and removed from treatment zone 32, rivets 206 and mesh 214 remain attached to plaque 82. Mesh 214 completely biodegrades or dissolves within about 30 minutes. Thereafter, rivets 206 remain anchored in plaque 84.

As previously mentioned, balloon 84 can include a tubular sheath which covers balloon outer surface 202 when balloon 84 is being navigated through the bodily lumen. The sheath can protect mesh 214 and rivets 206 and prevents mesh 214 and rivets 206 from detaching prematurely from balloon 84.

In some aspects of the method of FIG. 14 described above, the treatment zone is treated by simultaneously cooling (without cryoablation) and providing temporary structural support. Structural support is provided by plaque which has been temporarily frozen or hardened as a result of cooling. The method makes use of material (i.e., plaque) which is already present in the treatment zone to form the structural support. Thus, it is possible to provide support to the treatment zone without introducing a synthetic or foreign material into the treatment zone.

Advantages arising from simultaneously cooling and providing structural support can be accomplished in other ways. For example, a temporary support structure, which has been cooled to a temperature that will not result in cryoablation, can be introduced into the treatment zone. Such a method can make use of material added to the treatment zone to form the structural support.

Referring again to FIG. 1, material can be added to the treatment zone for the purpose of providing structural support and cooling. In the following method description, reference will be made to other figures that illustrate an exemplary catheter for performing the method. It is to be understood that the method is not limited to the illustrated catheter.

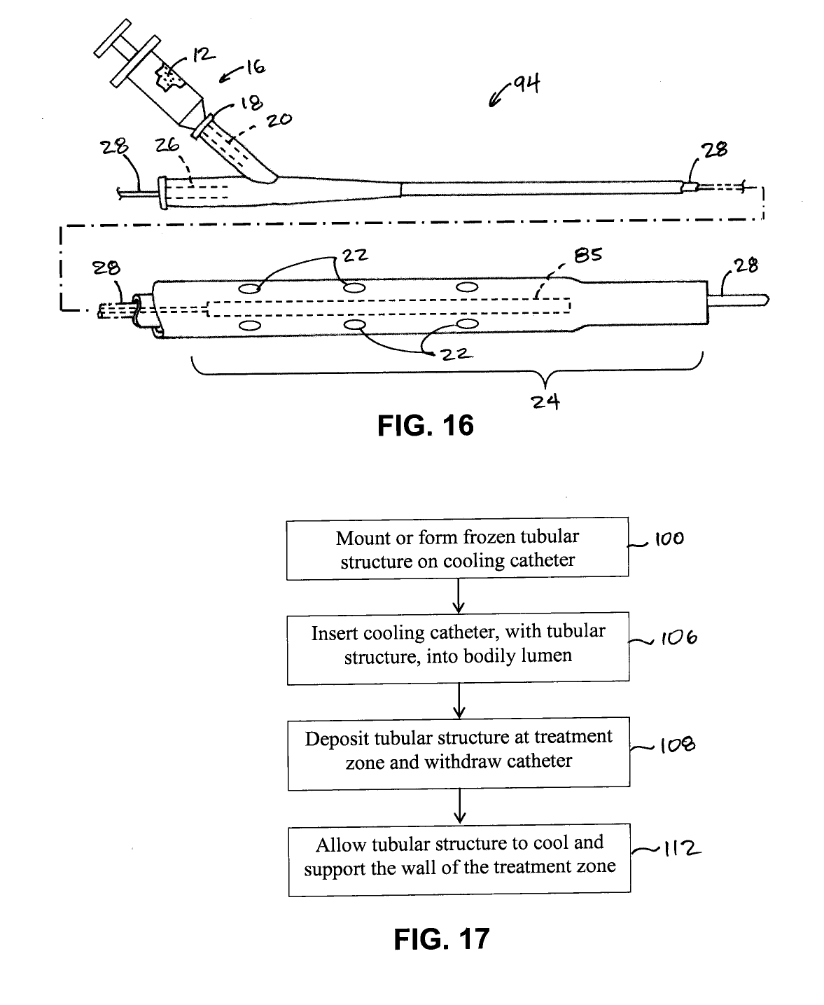

In block 10 (FIG. 1), formulation 12 is loaded into cooling catheter 94 (FIG. 16). Formulation 12 is capable of freezing at a temperature that is above the damage threshold of the wall of the treatment zone. Again, the term “damage threshold” is the temperature which will permanently destroy or damage tissue. Distal end segment 24 of catheter 94 is configured to change temperature. Specifically, distal end segment 24 is configured to drop to a temperature that will freeze formulation 12. Distal end segment 24 includes cooling and temperature control system 85. Cooling and temperature control system 85 is configured for cooling without cryoablation. Cooling and temperature control system 85 is configured to bring the outer surface of distal end segment 24 to a freezing temperature of formulation 12. The freezing temperature can be between about 0° C. and about −30° C., or between about −20° C. and about −30° C., or between about 0° C. and about −20° C., or between about −10° C. and about −20° C., or between about 0° C. and about −10° C., or between about 0° C. and about −5° C. Here, “about 0° C.” encompasses −1° C. to 1° C. Various cryoablation catheter cooling systems known in the art can be altered (such as by using a different refrigerant fluid) to make cooling and temperature control system 85.

In block 34 (FIG. 1), formulation 12 is ejected out of formulation outlet apertures 22 of cooling catheter 94 and onto wall 36 of treatment zone 32, similar to what was shown in FIG. 4A. Next, in block 46, formulation 12 is allowed to solidify on wall 36. For example, after formulation 12 is ejected, distal end segment 24 of catheter 94 (FIG. 16) is cooled to a temperature which freezes or solidifies formulation 12 but does not cryoablate wall 36. The solidified formulation can form a tube without fenestrations, similar to FIG. 5, or with fenestrations, similar to FIG. 6 or 11. In block 48, the solidified formulation 12 remains at treatment zone 32 and is allowed to support wall 36 of treatment zone 32, similar to FIG. 4B. For example, catheter 94 can be pulled out of the bodily lumen so that blood or other bodily fluids can flow through fluid passageway 38 within the solidified formulation. Thereafter, the solidified formulation can melt away and be resorbed by the body. Due heat transfer from surrounding tissue, the solidified formulation can melt completely within about 30 minutes, or within about 20 minutes, or within about 10 minutes, or without about 5 minutes.

For example, formulation 12 can be an aqueous solution. Formulation 12 can be an isotonic saline solution. The solution can be loaded into cooling catheter 94 (block 10 in FIG. 1), then ejected from catheter 94 (block 34), then cooled to solidify on wall 36 of treatment zone 32 (block 46), and then allowed to support wall 36 (block 48) until it melts (within any of the time periods specified above) due to heat transfer from surrounding tissue. To freeze the solution in block 46, distal end segment 24 of catheter 94 (FIG. 16) is cooled to a temperature equivalent to the freezing temperature of the solution. Freezing creates a support structure that is at a freezing temperature below about 25° C., and more specifically about 0° C. The freezing temperature will depend on the composition of the solution, such as saline concentration. For example, distal end segment 24 can be cooled to a freezing temperature between about 0° C. and about −30° C., or between about −20° C. and about −30° C., or between about 0° C. and about −20° C., or between about −10° C. and about −20° C., or between about 0° C. and about −10° C., or between about 0° C. and about −5° C. Here, “about 0° C.” encompasses −1° C. to 1° C.

In situ formation of the structural support for the bodily lumen, such as described in all embodiments above, can provide numerous advantages. For example, after formulation 12 is ejected out of outlet apertures 22, catheter can be repositioned to another treatment zone elsewhere in the bodily lumen. Thus, it is possible use a single catheter to treat multiple discrete lesions of varying percent stenosis (varying degree of blockage) along a bodily lumen such as a blood vessel. At each location, the solidified structural support fits the shape and size of the bodily lumen at that location, which is advantageous in cases of eccentric lesions which constitute a majority of coronary artery disease lesion cases. Many bodily lumens, such as coronary arteries, taper or have length-dependent diameters. In situ formation permits treatment with varying diameters along the length of the bodily lumen.

Another way of simultaneously cooling and providing structural support to treatment zone 32 is to introduce a preformed tubular structure that has been temporarily frozen before introduction into treatment zone 32. In this context, the term “preformed” means that the structural support is created outside of the patient's body.

In the method of FIG. 17, the treatment zone can be treated by simultaneously cooling (without cryoablation) and providing temporary structural support using a preformed tubular structure. The tubular structure is frozen while outside of the patient, then transported on a cooling catheter into the patient, then deposited in the treatment zone, and then allowed to melt in the treatment zone due to heat transfer from surrounding tissue. In the following description of the method of FIG. 17, reference will be made to other figures that illustrate an exemplary catheter for performing the method. It is to be understood that the method of FIG. 17 is not limited to the illustrated catheter.

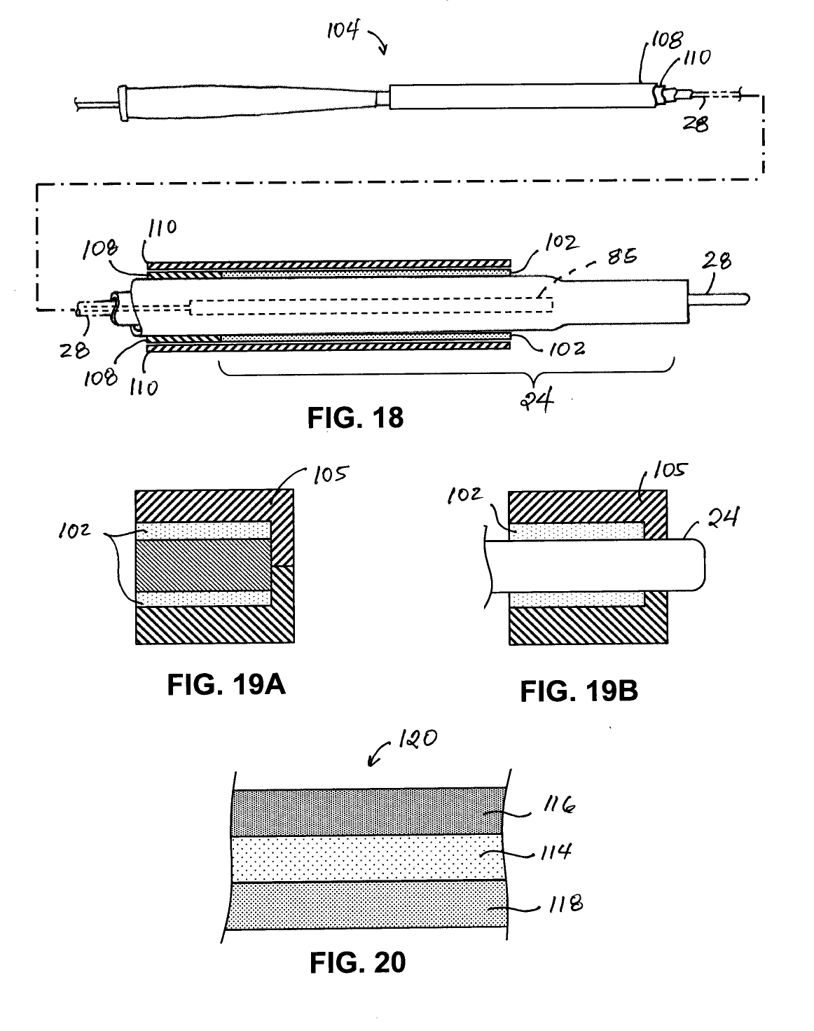

In block 100, tubular structure 102 is either mounted on or formed directly on distal end segment 24 of cooling catheter 104 (FIG. 18). Distal end segment 24 is configured to change temperature. Specifically, distal end segment 24 is configured to drop to a temperature that is at or below the freezing temperature of formulation used to make tubular structure 102. Distal end segment 24 includes cooling and temperature control system 85. Cooling and temperature control system 85 is configured for cooling without cryoablation. Cooling and temperature control system 85 is configured to bring the outer surface of distal end segment 24 to a freezing temperature of formulation used to make tubular structure 102. The freezing temperature can be between about 0° C. and about −30° C., or between about −20° C. and about −30° C., or between about 0° C. and about −20° C., or between about −10° C. and about −20° C., or between about 0° C. and about −10° C., or between about 0° C. and about −5° C. Here, “about 0° C.” encompasses −1° C. to 1° C. Various cryoablation catheter cooling systems known in the art can be altered (such as by using a different refrigerant fluid) to make cooling and temperature control system 85.

For example, as shown in FIG. 19A, a bioresorbable formulation can be introduced into mold 105 that is cooled to create frozen tubular structure 102. The bioresorbable formulation can be an aqueous solution. The bioresorbable formulation can be an isotonic saline solution. Mold 105 is configured to bring the surfaces of the mold cavity to a freezing temperature of the formulation which is used to make tubular structure 102. The freezing temperature can be any of the temperatures or temperature ranges listed above. When bioresorbable formulation is introduced into the mold cavity, bioresorbable formulation freezes and tubular structure 102 is formed. The mold cavity and the resulting frozen tubular structure can be similar in shape to tube 40 of any of FIGS. 5, 6, and 11, or it can have a different configuration. After freezing, the tubular structure is removed from the mold and then mounted onto distal end segment 24 of cooling catheter 104.

Alternatively, as shown in FIG. 19B, distal end segment 24 of cooling catheter 104 can be held in the mold cavity so that when bioresorbable formulation is introduced into the mold cavity, bioresorbable formulation freezes and tubular structure 102 is formed directly on distal end segment 24.

Alternatively, a bioresorbable formulation in liquid form can be applied onto distal end segment 24 of cooling catheter 104 without using a mold. The bioresorbable formulation can be an aqueous solution. The bioresorbable formulation can be an isotonic saline solution. Distal end segment 24 causes the formulation to freeze, which creates a frozen tubular structure directly on distal end segment 24 of cooling catheter 104. The tubular structure can be similar in shape to tube 40 of any of FIGS. 5, 6, and 11, or it can have a different configuration.

Distal end segment 24 is maintained at a temperature at or below the freezing temperature of the bioresorbable formulation and that is above the damage threshold of the treatment zone. The freezing temperature will depend on the composition of the solution, such as saline concentration. For example, distal end segment 24 of cooling catheter 104 (FIG. 18) can be cooled to a freezing temperature within any of the temperature ranges mentioned above for cooling catheter 94 (FIG. 16) and mold 105.

In block 106 (FIG. 17), after tubular structure 104 is mounted or formed on catheter 104, catheter is inserted into the bodily lumen. In block 108, when distal end segment 24 reaches the treatment zone, tubular structure 102 is deposited in the treatment zone and catheter 104 is withdrawn from the treatment zone.

Cooling catheter 104 includes cover sheath 108 and inner member 110 (FIG. 18). Cover sheath 108 is a tube that covers frozen tubular structure 102 when distal end segment 24 is pushed through the bodily lumen. Cover sheath 108 can help keep tubular structure 102 frozen during transport to treatment zone 32. Optionally, when distal end segment 24 reaches treatment zone 32, cover sheath 108 can be retracted away from distal end segment 24 to expose tubular structure 102. In addition or alternatively, inner member 110 moves into distal end segment 24 and pushes frozen tubular structure 102 off distal end segment 24 after distal end segment 24 reaches treatment zone 32. Inner member 110 can be a tube or rod disposed within cover sheath 108. Next, catheter 104 can be withdrawn from the bodily lumen to allow blood or other bodily fluid to pass through a fluid passageway in the center of tubular structure 102.

In block 112 (FIG. 17), after frozen tubular structure 102 is deposited at treatment zone 32, tubular structure 102 provides cooling and structural support to the wall of the treatment zone. Thereafter, tubular structure 102 melts away. Tubular structure 102 can melt completely within about 30 minutes, or within about 20 minutes, or within about 10 minutes, or without about 5 minutes.

In the method of FIG. 17, tubular structure 102 is performed. That is, tubular structure 102 is formed outside of the patient and later deposited in treatment zone 32. After being deposited in the treatment zone, tubular structure 102 melts due to an increase in temperature.

Alternatively, a preformed tubular structure can be made of a relatively fast biodegradable polymer composition. The preformed polymer tubular structure can be deposited in the treatment zone using any of the cooling catheters described above, and it can be deposited using a catheter that is not capable of cooling. The preformed polymer tubular structure can be deposited in a cooled or not-cooled state in the treatment zone.

For example, the preformed polymer tubular structure can be made from a formulation containing PEG or a PEG-based polymer. The formulation can be molded or caste to create a tubular shape, similar to what is shown in FIG. 5, 6, or 11. After its formation, the preformed tubular structure is mounted onto a catheter for transport into the treatment zone. As a further example, the formulation can be a blend of a therapeutic agent and either PEG or a PEG-based polymer. A PEG-based polymer is a polymer which has been modified using PEG. A PEG-based polymer can be block copolymer that includes PEG. The therapeutic agent can be in the form of nanoparticles, or the therapeutic agent can be encapsulated within nanoparticles, such as polyanhydride nanoparticles. The therapeutic agent is released as the PEG biodegrades. For example, a PEG tubular structure can biodegrade and disintegrate completely at a time after being deposited in the treatment zone of the bodily lumen. The time can be within the range of about 7 days to about 30 days. Alternatively, the time can be within the range of about 7 days to about 21 days, or the range of about 7 days to about 14 days. Thus, the therapeutic agent can be released over a period time of about 7 days to about 30 days, or about 7 days to about 21 days, or about 7 days to about 14 days.

As shown in FIG. 20, the blend of therapeutic agent and PEG or the blend of therapeutic agent and PEG-based polymer can be a layer 114 contained between two polymer layers 116 and 118. Outer polymer layers 116 and 118 biodegrade over a greater period of time than layer 114. Outer layer 116 can be made of PLA, and outer layer 118 can be made of polyglycolic acid (PGA). Together, layers 114, 116 and 118 form sheet material 120 used to make the walls of a preformed tubular structure, which can be similar in shape to tube 40 shown in any of FIGS. 5, 6, and 11. Optionally, fenestrations can be created by using a laser or knife to cut away material. After the preformed tubular structure is deposited in a treatment zone, PEG layer 114 biodegrades, which releases the therapeutic agent. Thereafter, outer layers 116 and 118 completely biodegrade over a period of time after being deposited in treatment zone of the bodily lumen. The period of time can be within the range of about 3 months to about 6 months.

In any of the embodiments described above, the formulation used to make the tubular structure (either preformed outside of the patient or formed in situ inside the patient) can include a therapeutic agent. For example, the therapeutic agent can be in the form of nanoparticles or encapsulated in polymer nanoparticles, such as in polyanhydride nanoparticles.

As used herein, the term “nanoparticle” encompasses coarse, fine, and ultrafine nanoparticles. A nanoparticle can have a diameter between 2,500 and 10,000 nanometers (for coarse nanoparticles), between 100 and 2,500 nanometers (for fine nanoparticles), or between 1 and 100 nanometers (for ultrafine nanoparticles).

In any of the embodiments described above, the therapeutic agent can be an antiproliferative, antineoplastic, anti-inflammatory, antiplatelet, anticoagulant, antifibrin, antithrombin, antimitotic, antibiotic, antiallergic, or antioxidant substance. Examples of therapeutic agents include without limitation sirolimus (rapamycin), everolimus, zotarolimus, Biolimus A9, AP23572, tacrolimus, pimecrolimus and derivates or analogs or combinations thereof.

While several particular forms of the invention have been illustrated and described, it will also be apparent that various modifications can be made without departing from the scope of the invention. It is also contemplated that various combinations or subcombinations of the specific features and aspects of the disclosed embodiments can be combined with or substituted for one another in order to form varying modes of the invention. Accordingly, it is not intended that the invention be limited, except as by the appended claims.

1. A method of treating a bodily lumen, the method comprising:
forming a structural support in situ within a treatment zone of a bodily lumen.

2. The method of claim 1, wherein forming the structural support includes ejecting a formulation through a lumen of a catheter and onto a wall of the treatment zone, followed by solidifying the ejected formulation on the wall of the treatment zone.

3. The method of claim 2, wherein the formulation includes a bioresorbable polymer and optionally a therapeutic agent.

4. The method of claim 3, wherein the bioresorbable polymer is a blend of at least one polymer and dimethyl sulfoxide (DMSO), and solidifying the ejected formulation includes allowing DMSO to exchange with an aqueous medium, wherein the at least one polymer solidifies as a result of the DMSO exchange.

5. The method of claim 4, wherein the at least one polymer includes polycaprolactone (PCL) and nanoparticles of poly(lactic acid) (PLA), and the PLA nanoparticles in PCL solidify as a result of the DMSO exchange.

6. The method of claim 2, wherein the formulation includes at least one photocrosslinkable polymer, and solidifying of the ejected formulation includes delivering optical radiation to the photocrosslinkable polymer in the treatment zone, wherein the at least one photocrosslinkable polymer increases in hardness as a result of the optical radiation.

7. The method of claim 6, wherein the at least one photocrosslinkable polymer includes any one or both of poly(lactic acid) diacrylate and poly(ethylene glycol) diacrylate.

8. The method of claim 2, wherein forming the structural support includes ejecting the formulation out of apertures formed through the catheter to form the structural support in situ, the apertures are arranged in a pattern on the catheter, and the structural support has the same pattern as the pattern on the catheter.

9. The method of claim 2, further comprising allowing a body fluid to pass through the treatment zone during any of ejecting the formulation and solidifying the ejected formulation.

10. The method of claim 2, wherein the formulation includes isotonic saline, and solidifying the ejected formulation includes freezing the isotonic saline in the treatment zone.

11. The method of claim 10, wherein freezing of the isotonic saline includes cooling the catheter to a temperature above a damage threshold of tissue in the treatment zone.

12. The method of claim 1, wherein forming of the structural support includes cooling plaque present in the treatment zone, and the cooling causes the plaque to increase in hardness.

13. The method of claim 12, wherein forming the structural support further includes compressing the plaque before or during cooling of the plaque.

14. The method of claim 12, wherein cooling of the plaque causes the plaque to increase in hardness without cyroablating tissue surrounding the plaque.

15. The method of claim 12, wherein cooling the plaque includes causing a catheter adjacent the plaque to drop to a temperature which is above a damage threshold of tissue in the treatment zone and which causes the plaque to increase and hardness.

16. The method of claim 1, wherein the forming of the structural support includes introducing an additive to plaque present in the treatment zone, and the additive causes formation of a hardened composite of the plaque and the additive.

17. The method of claim 16, wherein the additive is any one or a combination of two or more of fibrin glue, isopropyl cyanoacrylate, carboxymethyl cellulose, hydroxypropyl methylcellulose, and fibers made of bioresorbable polymer.

18. The method of claim 1, wherein the forming of the structural support includes pressing a plurality of bioabsorbable polymeric nanoparticles onto plaque present in the treatment zone, and the bioabsorbable polymeric nanoparticles cause the plaque to increase in hardness.

19. The method of claim 1, wherein the forming of the structural support includes anchoring a plurality of rivets into plaque present in the treatment zone, and the rivets cause the plaque to increase in hardness.

20. The method of claim 1, wherein the bodily lumen is a blood vessel.

21. A method of treating a bodily lumen, the method comprising:
cooling and structurally supporting a treatment zone of a bodily lumen, wherein the cooling and supporting are performed simultaneously.

22-28. (canceled)

29. An endoprosthesis comprising:
a support structure made of a frozen formulation having a freezing temperature below about 0° C.

30-31. (canceled)

32. A system for treating a bodily lumen, the system comprising:
the endoprosthesis of claim 29; and
a catheter configured to carry the structural support at a temperature at or below the freezing temperature.

33. An endoprosthesis comprising:
a structural support made of a bioresorbable formulation including a polymer selected from the group consisting PEG (polyethylene glycol) and a PEG based polymer.

34-37. (canceled)

38. A method of treating a bodily lumen, the method comprising:
depositing the structural support of claim 33 in a treatment zone of a bodily lumen; and
allowing the bioresorbable formulation to biodegrade completely at a time after the depositing, the time being within the range of about 7 days to about 30 days.

39. A catheter comprising:
an inflatable balloon; and
a plurality of bioabsorbable rivets carried on an outer surface of the balloon, each rivet including a tip and a base wider than the tip, the tips facing outward from the outer surface, each rivet configured to detach from the balloon when the tip is pressed into tissue.

40-41. (canceled)
